(19) United States
(12) Patent Application Publication
(10) Pub. No.: US 2017/0056459 A1
(43) Pub. Date: Mar. 2, 2017

(54) Compositions and Methods for Aiding Phase 3 Recovery After Anesthesia

(71) Applicant: Fast Track Recovery, LLC, Palisades, NY (US)

(72) Inventor: Nadine J. Keegan, Palisades, NY (US)

(73) Assignee: Fast Track Recovery, LLC, Palisades, NY (US)

(21) Appl. No.: 14/840,159

(22) Filed: Aug. 31, 2015

(51) Int. Cl. A61K 36/064; A61K 31/4045; A61K 31/198; A61K 31/375; A61K 9/00; A61K 31/122; A61K 31/09; A61K 36/16; A61K 35/745; A61K 35/747; A61K 31/522; A61K 36/9068

(52) U.S. Cl. CPC A61K 36/064; A61K 31/522; A61K 31/4045; A61K 31/198; A61K 31/375; A61K 36/9068; A61K 31/122; A61K 31/09; A61K 36/16; A61K 35/745; A61K 35/747; A61K 9/0053

(57) ABSTRACT

Compositions and methods for aiding Phase 3 recovery from post anesthesia and/or post colonoscopy procedure symptoms are disclosed.

### CROSS-REFERENCE TO RELATED APPLICATIONS

Not Applicable

### FIELD OF THE INVENTION

The invention relates to compositions in the form of dietary supplements that improve the “PHASE 3” recovery from anesthesia.

### BACKGROUND OF THE INVENTION

Recovery from anesthesia can be divided into different phases. The first phase, begins in the Operating Room when anesthetic agent administration is discontinued, and encompasses a return to minimal awareness, ability to maintain a patent airway, spontaneous and effective respiration, and a return to baseline vital signs (i.e, until the recovery of the protective reflexes and motor function” (Ref. Handbook of Ambulatory Anesthesia edited by Rebecca S. Twersky, Beverly K. Philip 2008) pg 355). These criteria are often used to establish admission to the recovery room or post-anesthesia care unit) (PACU), where Phase 1 recovery continues. Phase 1 Recovery is completed when the patient is suitable for transfer either to a hospital bed, or to a Phase II recovery unit. Criteria for Phase 1 Recovery and discharge from the PACU is often evaluated using the Aldrete Scoring system. The Aldrete Score is utilized as described in the chart below. A score of greater than or equal to 9 is often used as criteria for discharge.

The ‘modified’ Aldrete Scale

| | | | |
|---|---|---|---|
| Respiration | 2 | 1 | 0 |
| | Able to take | Dyspnea/Shallow | Apnea |
| | a deep breath | breathing | |
| | and cough | | |
| O₂ Saturation | 2 | 1 | 0 |
| | Maintains >92% | Needs O₂ | Saturation <90% |
| | on room air | inhalation to | even with |
| | | maintain O₂ | supplemental O₂ |
| | | saturation >90% | |
| Consciousness | 2 | 1 | 0 |
| | Fully Awake | Arousable on | Not responding |
| | | calling | |
| Circulation | 2 | 1 | 0 |
| | BP ± 20 | BP ± 20-50 | BP ± 50 |
| | mmHg pre op | mmHg pre op | mmHg pre op |
| Activity | 2 | 1 | 0 |
| | Able to move 4 | Able to move 2 | Able to move 0 |
| | extremities | extremities | extremities |
| | voluntarily | voluntarily | voluntarily |
| | or on command | or on command | or on command |

Phase 2 Recovery focuses on pain control and comfort. An ambulatory surgery patient is discharged to home from Phase 2 Recovery after he/she experiences minimal pain, nausea, vomiting or dizziness, is often tolerating oral liquids and continues to achieve an Aldrete Score of 9 or 10. Intermediate recovery (Phase 2) is the period during which the criteria for discharge from the ambulatory surgical unit are obtained.” (Twersky, pg 355)

Frequently, ambulatory surgery patients are transferred directly to Phase II recovery, bypassing the more costly PACU (Fast-Tracking, Twersky pg 356). Suitability for direct admission to Phase II recovery depends significantly on the anesthetic administered and the ability of the patient to rapidly recovery from the anesthetic effects and achieve Phase 1 recovery criteria while still in the operating room.

The third, and longest phase of recovery begins upon discharge from Phase 2 recovery. Late recovery (phase 3) lasts for several days or even weeks and continues until the patient is back to their preoperative functional status and is able to resume their daily activities and ends when the patient's functions of daily living and physiology are completely normalized. For ambulatory surgery patients, this third phase of recovery takes place entirely at home. (Ref. Handbook of Ambulatory Anesthesia edited by Rebecca S. Twersky, Beverly K. Philip 2008). The present invention is focused solely on Phase 3 recovery.

A number of patents have described compositions and treatments to accelerate Phase 1 recovery as well as Phase 2 Recovery, but have not addressed Phase 3. More specifically, Phase 1 and 2 recovery is addressed in U.S. Pat. No. 6,025,362, where dialkyl substituted xanthines are utilized, specifically water soluble theophylline compounds such as aminophylline. These compounds were tested in animal models via intravenous administration only. The method of the invention in the above patent shortens the time required for substantially complete recovery of psychomotor functions, addressing primarily Phases 1 and 2, but not Phase 3 of recovery.

U.S. Pat. No. 6,025,362 evaluates the degree or level of post-anesthesia recovery using the Aldrete Score, relevant only in Phase 1 and Phase 2 recovery settings, the Ramsay score, determining the level of sedation, and the Fukunaga score, also relevant to Phase 1 and Phase 2 Recovery. Again, these do not address issues in Phase 3 recovery.

US 2015/0196249 (equivalent to WO2012031125 A2) relates to compositions comprising anesthesia-reversing agents and methods of their use for facilitating emergence of a subject from anesthesia-induced unconsciousness and restoring the subject back to consciousness and cognitive function. This invention can also be used for prophylaxis treatment where the subject is at increased risk of post-operative delirium after general anesthesia, which occurs by definition during Phase 1 of recovery. Thus, this document addresses Phases I and II of recovery from anesthesia only. The anesthesia reversing agent described in this document can be selected from any of methylphenidate (MPH), amphetamine, modafinil, amantadine, caffeine, or analogues or derivatives thereof or combinations of these agents with each other. The focus of this document is directed towards facilitating emergence or awakening from anesthesia and is administered during or at the conclusion of an anesthetic. One aspect of that disclosure relates to use of caffeine as an anesthesia-reversing agent to rapidly reverse a subject's unconsciousness by general anesthesia, decreasing the time to Phase 1 recovery.

Phase 3 begins upon discharge from the Phase 2 recovery area either to home, in the case of ambulatory surgery patients, or to an inpatient hospital bed in the case of patients who need one or more days of hospitalization after surgery. Phase 3 continues until the patient is back to his/her pre-procedure state, or renormalizes. Phase 3 may take less than one or more than several weeks to complete depending on the length and complexity of the surgery and the patient's pre-surgical medical status. Activities of daily living that require cognition and energy, such as work and exercise are resumed. Diet and gastrointestinal function are renormalized. Pain medications taken post operatively may contribute to prolonged Phase 3 Recovery as can the lingering effects of anesthesia and its metabolites as well as surgical wound healing. Antibiotics, often administered in the peri-operative period may disturb GI flora and cause diarrhea, constipation and/or abdominal pain.

It has long been known that after the use of antibiotics (that deplete the normal intestinal flora resulting in symptoms such as bloating and diarrhea) that the ingestion of yogurt with live cultures and other probiotic supplements aid in the restoration of more normal GI and bowel function. See, for example, U.S. Pat. No. 5,902,578.

It is also known that vitamin C, a water soluble essential vitamin, promotes wound healing as it is a cofactor in collagen synthesis. Vitamin C has anti-oxidant properties and may also promote increased blood flow in instances of impaired blood flow (Stamatelopoulos K S et al; Oral administration of ascorbic acid attenuates endothelial dysfunction after short-term cigarette smoking; Int J Vitam Nutr Res. 2003 November; 73(6):417-22).

L-theanine is a non-essential amino acid that promotes relaxation and anxiolysis without drowsiness. It may also promote improved sleep quality. (See (a) Lu et al; The acute effects of L-theanine in comparison with alprazolam on anticipatory anxiety in humans, (2004) The acute effects of L-theanine in comparison with alprazolam on anticipatory anxiety in humans. Human Psychopharmacology, 19 7: 457-465. doi:10.1002/hup.611; (b) Kimura K et al; Biol Psychol. 2007 January; 74(1):39-45. Epub 2006 Aug. 22, L-Theanine reduces psychological and physiological stress responses; and (c) Alten Med Rev. 2011 December; 16(4):348-54, The effects of L-theanine (Suntheanine®) on objective sleep quality in boys with attention deficit hyperactivity disorder (ADHD): a randomized, double-blind, placebo-controlled clinical trial.)

Ginger is used as a preventative treatment for nausea. and may also decrease inflammation. (See Ernst et al, Br J Anaesth. 2000 March; 84(3):367-71, Efficacy of ginger for nausea and vomiting: a systematic review of randomized clinical trials; Chaiyakunapruk, et al; Am J Obstet Gynecol. 2006 January; 194(1):95-9, The efficacy of ginger for the prevention of postoperative nausea and vomiting: a meta-analysis.)

Ginkgo Biloba promotes memory and cognitive speed.

(See (a) J Psychiatr Res. 2012 June; 46(6):716-23. doi: 10.1016/j.jpsychires.2012.03.003. Epub 2012 Mar. 27, Ginkgo biloba extract EGb 761® in dementia with neuropsychiatric features: a randomised, placebo-controlled trial to confirm the efficacy and safety of a daily dose of 240 mg; and (b) Altern, et al, J Complement Med. 2000 June; 6(3):219-29; An examination of the efficacy of Ginkgo biloba extract EGb761 on the neuropsychologic functioning of cognitively intact older adults.)

Curcumin is believed to reduce inflammation and may act as an anti-oxidant and promote post-operative pain relief. (See (a) DiSilvestro et al, Nutr J. 2012 Sep. 26; 11:79. doi: 10.1186/1475-2891-11-79, Diverse effects of a low dose supplement of lipidated curcumin in healthy middle aged people and (b) Agarwal et al, Surg Endosc. 2011 December; 25(12):3805-10. doi: 10.1007/s00464-011-1793-z. Epub 2011 Jun. 14, Efficacy of turmeric (curcumin) in pain and postoperative fatigue after laparoscopic cholecystectomy: a double-blind, randomized placebo-controlled study.)

Caffeine has been known to be useful in an intravenous administration to facilitate the time to awaken from general anesthesia, as described in WO2012/031125. EP2108270 is directed to the use of tea based products having theanine and caffeine for enhancing mental alertness, while EP1393726 discusses the use of theanine to improve concentration and relaxation.

US 2010/0061969 mentions Coenzyme Q as an anti-fatigue agent. US 2008/0287520 discusses the use of melatonin prior to surgery in preventing postoperative complications.

Other prescription agents, none of which are suitable for a nutritional supplement product, which have been used for anesthesia rescue or anesthesia recovery accelerants (relating to Phase 1 recovery) have included intravenous aminophylline (U.S. Pat. No. 6,025,362); methylphenidate, amphetamine, modafinil, and amantadine (WO2012/031125); Nk-1 receptor antagonists (WO2005/082366); intravenous ribose (CA 2552529); intravenous bicarbonate (US 2009/0131338); modafinil (U.S. Pat. No. 7,528,172); and intravenous idebenone (US 2010/0099775).

Unfortunately, many of the symptoms of anesthesia Phase 3 recovery are not adequately addressed and patients must either simply contend with the undesirable after effects or treat their symptomology on an ad hoc basis.

### OBJECTS OF THE INVENTION

It is therefore an object of the invention to provide a nutritional supplement (and/or nutraceutical) formulation that speeds the recovery from Phase 3 symptoms associated with general anesthesia, intra-venous sedation, and/or colonoscopy procedures that may or may not involve intra-venous sedation.

It is a further object of the invention to provide a nutritional supplement (and/or nutraceutical) formulation that permits a patient to return to normal functioning sooner than without such nutritional supplement use due to relief of the Phase 3 symptoms associated with anesthesia or colonoscopy procedures.

Yet another object of the invention is to provide nutritional supplement (and/or nutraceutical) formulation that can be utilized by the patient without further medical supervision for the relief of lingering Phase 3 symptoms associated with anesthesia and/or colonoscopy procedures.

A still further object of the invention is a method of improving recovery from symptoms of anesthetic use and/or colonoscopy procedures by administration of the nutritional supplement and/or the nutraceutical formulation of the invention.

Still further objects of the invention will be appreciated by those of ordinary skill in the art.

While the present invention generally achieves the foregoing objects, it will be appreciated that some embodiments will achieve all of them, while other embodiments will achieve some, but not all. In addition, different embodiments will achieve the objects in varying degrees, some fully achieving some objects while other objects to a lesser extent.

### SUMMARY OF THE INVENTION

The present invention compositions, comprises at least (a) caffeine (for daytime use) or melatonin (for evening/bedtime use) each combined with at least a probiotic. Each combination may optionally further include one or more of the following: Vitamin C, L-theanine, Ginger, Coenzyme Q10, Curcumin, and Gingko Biloba; and may further include other suitable agents that are not incompatible therewith. The compositions address issues and symptoms of Phase 3 recovery after anesthesia. The invention is intended for use after discharge from both Phase 1 and Phase 2 recovery areas (PACUs). This invention, which is administered solely by mouth, is most likely to be used at home following ambulatory surgery during Phase 3 recovery, and not at earlier phases of such recovery.

### DETAILED DESCRIPTION OF THE INVENTION

The present invention is directed to formulations that contain at least a probiotic, and one of caffeine (for daytime administration) or melatonin (for evening or bedtime administration). In addition, optional components (to each of the foregoing) include one or more of L-theanine, Vitamin C, Ginger, and Ginkgo biloba, Coenzyme Q and Curcumin. The compositions are for use in aiding the Phase 3 recovery from anesthesia, either general or otherwise, and most preferably from general anesthesia. For purposes of the present invention, “anesthesia” may be strictly defined to exclude “iv sedation” used for certain medical procedures, but where desired may include such “iv sedation”. Where such “iv sedation” is specifically intended to be excluded, “general anesthesia” will be utilized specifically used to denote the more limited definition.

For all chemical components, the amount of such component whether in mg per dosage unit or parts by weight, or w/w %, are all expressed based on the free base or free acid of the chemical component in question. Thus, if caffeine is discussed and the actual entity being used is caffeine hydrochloride, all weight and parts and % are based on the amount of anhydrous caffeine free base (mol. wt. 194.19) and the actual amount of the anhydrous hydrochloride salt (mol. wt. 212.19) will be adjusted to provide the stated amount of free base. (212.19/194.19=1.093, so that amounts stated anhydrous caffeine free base are increased by 9.3% if the anhydrous caffeine hydrochloride salt is used.) Similar calculations known to those of ordinary skill in the art are applied to accommodate the use of other salts and/or hydrated forms.

Dosage forms intended for daytime use are required to include caffeine, while those intended for bedtime or evening use are required to have melatonin instead of caffeine. When caffeine is used, the caffeine may be selected from the free base or any suitable orally ingestible salt form thereof, and any of these may be in the anhydrous form or in any acceptable hydrate thereof. When caffeine is present it is used for adult dosing in amounts based on the free caffeine base, in amounts of from about 20 mg to about 200 mg per dose, preferably about 25 mg to about 175 mg per dose, more preferably about 50 mg to about 150 mg per dose, still more preferably about 75 mg to about 125 mg per dose, most preferably about 100 mg per dose, each based on the free caffeine regardless of the caffeine salt used. When melatonin is used, it is present in an amount based on the free melatonin base, regardless of the salt or free base used, in an amount of about 0.3 mg to about 10 mg per dose, preferably about 0.5 mg to about 7.5 mg per dose, more preferably about 1 mg to about 5 mg per dose, still more preferably about 2 mg to about 4 mg per dose, most preferably about 3 mg per dose.

The probiotic component can be chosen from a wide range of known probiotic materials. These include, without limitation, bifidobacterium breve, Bifidobacterium lactis, Lactobacillus acidophilus, Lactobacillus plantarum, Lactobacillus rhamnosus, and Saccharomyces boulardii, although any probiotic (inclusive of bacteria and yeasts) that is known to be acceptable for human ingestion and is known to beneficially repopulate the GI flora after antibiotic or cleansing prep use may be used as well. The probiotic may be used as a single entity or mixtures of two or more. Preferably, for post-GI cleansing preps such as those used in colonoscopy prep procedures, the probiotic component is one or more of Bifidobacterium breve, Bifidobacterium lactis, Lactobacillus acidophilus, Lactobacillus plantarum, Lactobacillus rhamnosus, more preferably a blend of all 5 thereof. Preferably for Phase 3 recovery post anesthesia, not associated with a GI-cleansing prep, the probiotic is Saccharomyces boulardii. In some variations, various mixtures of one or more probiotic bacteria and one or more probiotic yeasts may be preferred. The Saccharomyces boulardii, when used, is generally used; for adult dosing, in amounts of about 50 to about 500 mg/dose, preferably about 100 to about 400 mg/dose, more preferably about 150 to about 250 mg/dose, most preferably about 200 mg/dose. When other yeasts are used (such as Saccharomyces cerevisiae, Saccharomyuces pastorianus, Saccharomyces bayanus, etc., (see Probiotic yeasts: Anti-inflammatory potential of various non-pathogenic strains in experimental colitis in mice. World J Gastroenterol. 2010 May 7; 16(17): 2134-2145. Published online 2010 May 7. doi: 10.3748/wig.v16.i17.2134) in place thereof, similar dosings may be used. When combinations of yeasts are used, the total yeast content should also remain within these ranges. When the various bacteria are used, then can be used, for adult dosing, in amounts of from about 1 Billion to about 15 Billion Colony Forming Units (CFU) per dose, preferably about 2 Billion to about 14 Billion CFU/dose, more preferably about 4 Billion to about 13 Billion CFU/dose, still more preferably about 8 Billion to about 12 Billion CFU/dose, and most preferably about 10 Billion CFU/dose. Alternative bacteria can be used in the same amounts, although the total bacteria levels should also be within these ranges. Combinations of yeasts and bacteria can be used, provided the maximum bacterial dose is not exceeded and the maximum yeast dose is not exceeded. Non-limiting exemplary commercially available probitotics include FloraFIT Probiotics (DuPont Danisco) http://www.danisco.com/product-range/probiotics/florafitr-probiotics/.

Theanine is generally of the structural formula:

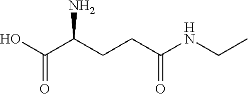

and L-theanine is used in the present invention. Either the free base or an ingestible salt thereof is acceptable, and if a salt is used, it is used in amounts that provide the dose of the free base expressed below. The L-theanine component is present for adult dosing, in an amount of about 75 mg to 300 mg per dose for the free base (or an amount of a salt that provides the amount of the free base), preferably about 100 mg to about 250 mg per dose. Particularly preferred compositions contain either about 100 mg/dose or 200 mg/dose for an adult dosing. Generally, preferred formulations for daytime use have about 100 mg of L-theanine per dose, while preferred formulations for bedtime use have about 200 mg/dose.

The Vitamin C component can be in the form of any orally ingestible form of vitamin C or any orally ingestible salt or ester or amide thereof. The vitamin C component (based on the non-salt, non-ester, non-amide free acid form) is present, for adult dosing, in the range of from about 25 mg to about 500 mg per dose, preferably about 50 mg to about 400 mg per dose, more preferably about 100 mg to about 250 mg per dose, more preferably about 125 mg to about 200 mg per dose, most preferably about 150 mg/dose.

The ginger component is generally present in the form of a powder extract and the powder is present generally, for adult dosing, in an amount of about 50 mg to about 1000 mg per dose, preferably 100 mg to 750 mg per dose, more preferably about 125 mg to about 500 mg per dose, more preferably about 150 mg to about 375 mg per dose, still more preferably about 175 mg to about 250 mg per dose, most preferably about 200 mg per dose.

The ginkgo biloba component is preferably present, for adult dosing, in an amount of about 30 mg to about 300×mg per dose, preferably about 60 mg to about 180 mg per dose, more preferably about 90 mg to about 150 mg per dose, most preferably about 120 mg per dose.

Optionally, coenzyme Q10 and Curcumin may be included, and preferably both are included in formulations intended as Phase 3 post anesthesia supplement. Formulations intended for post colonoscopy supplementation preferably omit both of these components. Nonetheless, where desired, either or both of these components may be included in the post colonoscopy supplement formulations and/or either or both of these components may be omitted from the formulations intended for Phase 3 post anesthesia supplements.

When coenzyme Q10 is used, it is present, for adult dosing, in amounts of from about 10 mg to about 200 mg per dose, preferably about 15 to about 150 mg per dose, more preferably about 20 mg to about 100 mg per dose, still more preferably about 25 mg to about 50 mg per dose, most preferably about 30 mg per dose.

When curcumin is used, it is preferably used, for adult dosing, in amounts of from about 20 mg to about 300 mg per dose, preferably about 30 mg to about 250 mg per dose, more preferably about 50 mg to about 200 mg per dose, still more preferably about 75 mg to about 150 mg per dose, most preferably about 100 mg per dose.

The respective formulations are prepared as follows: The various powders are blended and filled into capsules. Where the probiotic is not a powder, but a liquid, it may be used to granulate one or more of the respective powders and the granulate is filled into capsules. Alternatively, the respective components can be blended and granulated and compressed into tablets, with or without further excipients known in the pharmaceutical and nutriceutical arts. Such excipients may include, carriers, fillers, binders, disintegrants, lubricants, enteric coating materials, delayed release component materials, sustained release component materials, etc. as may be desired. A few inactive excipients may include, but are not limited to, microcrystalline cellulose, silicon dioxide, magnesium stearate and stearic acid. In one embodiment, the various components are simply blended and filled into capsules, preferably the capsules are Vegetable Capsules, but any suitable capsule is suitable.

The formulations are generally dosed as follows, although variations on the theme will be appreciated by those of ordinary skill. In addition, adjustments can be made for accommodating patient-patient variation in weight, degree of symptomology, etc. Generally, on the first day, once drinking fluids after either anesthesia or colonoscopy, 2 doses of the respective daytime formulation (post anesthesia daytime formulation if that is the procedure recovering from, or the post colonoscopy daytime formulation if that is the procedure recovering from) are taken, with an additional dose about every 4 hours until approximately 6 PM and then 1 dose of the respective bedtime formulation about 1 hour before bedtime. For each of the next three days, 1 dose of the respective daytime formulation is taken three times a day all before about 6 PM, preferably approximately evenly spaced apart, followed by 1 dose of the bedtime formulation about 1 hour before bed. Then, preferably for the next 6 days 1 dose of the respective daytime formulation twice a day each before about 6 PM and preferably approximately evenly spaced apart, followed by 1 dose of the respective bedtime formulation about 1 hour before bedtime.

The foregoing formulations are one embodiment of the invention. Alternate embodiments, such as sustained release formulations (tablets or capsules), rapid oral dissolution formulations, rapid dissolution formulations for dissolution into fluids to be consumed, enteric coated (tablets or capsules), etc will be well appreciated by those of ordinary skill in the art.

Having described the present invention, the following examples are presented to exemplify one or more aspects and/or embodiments, but do not limit the scope of the invention.

### EXAMPLES

### Example 1

A formulation of the invention, for adult dosing, intended for use as a daytime supplement in the Phase 3 recovery from anesthesia follows:

| | |
|---|---|
| 100 mg | Caffeine |
| 200 mg | Saccharomyces boulardii |

The above components are blended together and filled into hard gelatin capsules.

### Example 2

A formulation of the invention, for adult dosing, intended for use as a daytime supplement in the Phase iii recovery from anesthesia follows:

| | |
|---|---|
| 100 mg | Caffeine |
| 200 mg | Saccharomyces boulardii |
| 100 mg | L-theanine |
| 150 mg | Vitamin C |
| 200 mg | Ginger (powder extract) |
| 30 mg | Coenzyme Q10 |
| 100 mg | Curcumin |
| 120 mg | Ginkgo biloba |

The above components are blended together and filled into hard gelatin capsules.

### Example 3

A formulation of the invention, for adult dosing, intended for use as an approximate bedtime supplement in the Phase III recovery from anesthesia follows:

| | |
|---|---|
| 3 mg | melatonin |
| 200 mg | Saccharomyces boulardii |

The above components are blended together and filled into hard gelatin capsules.

### Example 4

A formulation of the invention, for adult dosing, intended for use as an approximate bedtime supplement in the Phase 111 recovery from anesthesia follows:

| | |
|---|---|
| 3 mg | melatonin |
| 200 mg | Saccharomyces boulardii |
| 100 mg | L-theanine |
| 150 mg | Vitamin C |
| 200 mg | Ginger (5:1 powder extract) |
| 30 mg | Coenzyme Q10 |
| 100 mg | Curcumin |
| 120 mg | Ginkgo biloba |

The above components are blended together and filled into hard gelatin capsules.

### Example 5

A formulation of the invention intended for use as a daytime supplement in the recovery from a colonoscopy procedure follows:

| | |
|---|---|
| 100 mg | Caffeine |
| 10 Billion | probiotic mix of 5 strains in a total bacterial |
| Colony | content of 10 Billion Colony Forming Units |
| Forming | per dosage having microencapsulated Bifidobacterium breve, |
| Units | Bifidobacterium lactis, Lactobacillus acidolpholus, |
| | Lactobacillus plantarum, and Lactobacillus rhamnosus. |

### Example 5

A formulation of the invention intended for use as a daytime supplement in the recovery from a colonoscopy procedure follows:

| | |
|---|---|
| 100 mg | Caffeine |
| 10 Billion | microencapsulated probiotic mix of 5 strains in a |
| Colony | total bacterial content of 10 Billion Colony Forming Units |
| Forming | per dosage having microencapsulated Bifidobacterium breve, |
| Unit | Bifidobacterium lactis, Lactobacillus acidolpholus, |
| | Lactobacillus plantarum, and Lactobacillus rhamnosus |
| 100 mg | L-theanine |
| 150 mg | Vitamin C |
| 200 mg | Ginger (5:1 powder extract) |
| 30 mg | Coenzyme Q10 |
| 100 mg | Curcumin |
| 120 mg | Ginkgo biloba |

The above components are blended together and filled into hard gelatin capsules.

### Example 7

A formulation of the invention intended for use as an approximate bedtime supplement in the recovery from a colonoscopy procedure follows:

| | |
|---|---|
| 3 mg | melatonin |
| 10 Billion | microencapsulated probiotic mix of 5 strains in a |
| Colony | total bacterial content of 10 Billion Colony Forming Units |
| Forming | per dosage having microencapsulated Bifidobacterium breve, |
| Units | Bifidobacterium lactis, Lactobacillus acidolpholus, |
| | Lactobacillus plantarum, and Lactobacillus rhamnosus |

The above components are blended together and filled into hard gelatin capsules.

### Example 8

A formulation of the invention intended for use as an approximate bedtime supplement in the recovery from a colonoscopy procedure follows:

| | |
|---|---|
| 3 mg | melatonin |
| 10 Billion | microencapsulated probiotic mix of 5 strains in a |
| Colony | total bacterial content of 10 Billion Colony Forming Units |
| Forming | per dosage having microencapsulated Bifidobacterium breve. |
| Units | Bifidobacterium lactis, Lactobacillus acidolpholus, |
| | Lactobacillus plantarum, and Lctobacillus rhamnosus |
| 100 mg | L-theanine |
| 150 mg | Vitamin C |
| 200 mg | Ginger (5:1 powder extract) |
| 30 mg | Coenzyme Q10 |
| 100 mg | Curcumin |
| 120 mg | Ginkgo biloba |

The above components are blended together and filled into hard gelatin capsules.

### Example 9

9 patients who underwent anesthetic procedures were surveyed as to their experience with recovery from anesthesia in the past. Each of the nine patients were administered a double dose of the daytime composition of the invention set forth below on being upgraded to Phase 3 of recovery, followed by an additional dose of the daytime composition (see below) every 4 hours thereafter until about 6 PM and about 4 hours later, a single dose of the nighttime composition (see below was administered). On awakening (day 2), then next morning a single dose of the daytime composition was administered every 4 hours until about 6 PM, and a single dose of the nighttime composition was administered about 4 hours later. The day 2 regimen was repeated for 5 additional days (total course of supplement was 7 days).

7 of those patients responded that they did receive anesthesia previously to the current anesthetic procedure and 2 indicated that they did not. The 7 patients who reported prior experience with anesthesia were surveyed and asked to rate their experience in the past concerning nausea, headache, anxiety, excessive sleepiness, worsened memory, difficulty sleeping, and symptoms form antibiotics, and to rate these as either no such symptom, mild, moderate, or severe. The results of that survey are reproduced below.

| | | None | Mild | Moderate | Severe | Total |
|---|---|---|---|---|---|---|
| Nausea | Counts | 3 | 1 | 1 | 2 | 7 |
| | % Within | 42.86% | 14.29% | 14.29% | 28.57% | 100.0% |
| Headache | Counts | 4 | 0 | 1 | 1 | 6 |
| | % Within | 66.67% | 0.00% | 16.67% | 16.67% | 100.0% |
| Anxiety | Counts | 5 | 1 | 1 | 0 | 7 |
| | % Within | 71.43% | 14.29% | 14.29% | 0.00% | 100.0% |
| Excessive | Counts | 2 | 3 | 1 | 1 | 7 |
| Sleepiness | % Within | 28.57% | 42.86% | 14.29% | 14.29% | 100.0% |
| Worsened | Counts | 5 | 1 | 0 | 1 | 7 |
| Memory | % Within | 71.43% | 14.29% | 0.00% | 14.29% | 100.0% |
| Difficulty | Counts | 4 | 2 | 0 | 0 | 6 |
| Sleeping | % Within | 66.67% | 33.33% | 0.00% | 0.00% | 100.0% |
| Symptoms | Counts | 4 | 0 | 1 | 0 | 5 |
| from | % Within | 80.00% | 0.00% | 20.00% | 0.00% | 100.0% |
| Anti- | | | | | | |
| biotics | | | | | | |

All 9 patients who were given the dietary supplement postoperatively were surveyed as to their most recent experience with anesthesia in the same manner as above concerning their prior experience with anesthesia. The results are reproduced below.

| | | None | Mild | Moderate | Severe | Total |
|---|---|---|---|---|---|---|
| Nausea | Counts | 6 | 2 | 0 | 1 | 9 |
| | % Within | 66.67% | 22.22% | 0.00% | 11.11% | 100.0% |
| Headache | Counts | 4 | 3 | 1 | 1 | 9 |
| | % Within | 44.44% | 33.33% | 11.11% | 11.11% | 100.0% |
| Anxiety | Counts | 7 | 1 | 1 | 0 | 9 |
| | % Within | 77.78% | 11.11% | 11.11% | 0.00% | 100.0% |
| Excessive Sleepiness | Counts | 4 | 3 | 2 | 0 | 9 |
| | % Within | 44.44% | 33.33% | 22.22% | 0.00% | 100.0% |
| Worsened Memory | Counts | 8 | 0 | 1 | 0 | 9 |
| | % Within | 88.89% | 0.00% | 11.11% | 0.00% | 100.0% |
| Difficulty Sleeping | Counts | 9 | 0 | 0 | 0 | 9 |
| | % Within | 100.00% | 0.00% | 0.00% | 0.00% | 100.0% |
| Symptoms from | Counts | 6 | 0 | 0 | 0 | 6 |
| Antibiotics | % Within | 100.00% | 0.00% | 0.00% | 0.00% | 100.0% |

As can be seen from a direct comparison of the corresponding portions of the two tables above, 5 of the 6 symptoms saw increases in the proportion of patients that reported no such symptom. Only “Headache” saw a reduction in the proportion of patients reporting no such symptom after receiving the supplement postoperatively.

The “daytime composition” used in the above study was:
a. Caffeine—100 mg./dose
b. Probiotic—10 Billion CFU/capsule 5 strain combination, microencapsulated, (Bifidobacterium breve, Bifidobacterium lactis, Lactobacillus acidolphillus, Lactobacillus plantarum. Lactobacillus rhamnosus) 4 Billion CFU/capsule
c. L-theanine—100 mg/dose
d. Vitamin C 150 mg/dose
e. Ginger—200 mg/dose and
f. Ginkgo Biloba—120 mg/dose

The “nighttime composition” used in the above study was:
a. Melatonin 3 mg/dose
b. Probiotic—10 Billion CFU/capsule 5 strain combination, microencapsulated, (Bifidobacterium breve, Bifidobacterium lactis, Lactobacillus acidolphillus, Lactobacillus plantarum, Lactobacillus rhamnosus) 4 Billion CFU/capsule
c. L-theanine—200 mg
d. Vitamin C 150 mg/dose
e. Ginger—200 mg/dose and
f. Ginkgo Biloba—120 mg/dose

What is claimed is:

1. A composition for use in promoting Phase 3 recovery from post-anesthesia symptoms and/or from post-colonoscopy procedure symptoms comprising:
(a) a component selected from Caffeine or a salt thereof or melatonin or a salt thereof; and
(b) an orally acceptable probiotic;

2. The composition of claim 1 further comprising at least one further component selected form the group consisting of
(c) L-theanine or a salt or ester or amide;
(d) Vitamin C or a salt or ester or amide thereof;
(e) Ginger (5:1 powder extract);
(f) Coenzyme Q10;
(g) Curcumin; and
(h) Ginkgo biloba.

3. The composition of claim 1 for daytime use wherein said component (a) is caffeine.

4. The composition of claim 1 for use in the evening or at or near bedtime wherein said component (a) is melatonin or a salt thereof.

5. The composition of claim 1 for use in the recovery from Phase 3 post anesthesia wherein at least one of said Coenzyme Q10 and said curcumin is present.

6. The composition of claim 1 for use in the Phase 3 recovery from post anesthesia wherein both of said Coenzyme Q10 and said curcumin are present.

7. The composition of claim 1 for use in the recovery from a colonoscopy procedure wherein at least one of said Coenzyme Q10 and said curcumin is absent.

8. The composition of claim 1 for use in the recovery from anesthesia wherein both of said Coenzyme Q10 and said curcumin are absent.

9. The composition of claim 1 wherein when caffeine is present it is present in the form of caffeine free base in an adult dosing amount of 20 mg to 200 mg or a corresponding amount of a pharmaceutically acceptable ingestible or nutriceutically acceptable ingestible salt thereof, and when melatonin is present, it is present in the form of the free base thereof in an adult dosing amount of about 0.3 mg to about 10 mg or a corresponding amount of a pharmaceutically acceptable ingestible or nutriceutically acceptable ingestible salt thereof.

10. The composition of claim 9 further comprising at least one further component in adult dosing amounts selected form the group consisting of
(c) about 75 mg to about 300 mg L-theanine or a corresponding amount of a pharmaceutically acceptable ingestible salt or ester or amide thereof;
(d) about 25 mg to about 500 mg Vitamin C or a corresponding amount of a pharmaceutically acceptable ingestible salt or ester or amide thereof;
(e) about 50 mg to about 1000 mg Ginger powder extract;
(f) about 10 mg to about 200 mg Coenzyme Q10;
(g) about 20 mg to about 300 mg Curcumin; and
(h) about 30 mg to about 300 mg Ginkgo biloba.

11. A method of aiding Phase 3 recovery from post-anesthesia symptoms and/or post-colonoscopy procedure symptoms comprising administering to a patient in need thereof a composition of claim 1.

12. The method of claim 11 comprising administering to said patient once said patient is drinking fluids, 2 doses of said composition, wherein said composition has caffeine present, and an additional dose of said composition having said caffeine every 4 hours until about 6 PM and then 1 dose of said composition having melatonin present at about 1 hour before bedtime, thereby closing day 1.

13. The method of claim 12 further comprising administering to said patient 1 dose of said composition having caffeine present three times a day approximately evenly spaced apart all before about 6 PM and 1 dose of said composition having melatonin present at about 1 hour before bedtime for 3 days beginning on day 2.

14. The method of claim 13 further comprising administering to said patient 1 dose of said composition having caffeine present two times a day approximately evenly spaced apart all before about 6 PM and 1 dose of said composition having melatonin present at about 1 hour before bedtime for at least 6 days, beginning on day 5.
